(19) United States
(12) Patent Application Publication
(10) Pub. No.: US 2007/0105115 A1
(43) Pub. Date: May 10, 2007

(54) Promoter of the human fatp5 gene and uses

(75) Inventors: Geneviève Martin, Loos (FR); Guy Augert, Lille (FR); Maryam Asfari, Savigny sur Orge (FR); Herve Dupont, Lyon (FR); Daniel Ruggiero, Gainte Consorce (FR); Philippe Durbin, Villeurbanne (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK SANTE, Lyon Cedex (FR)

(21) Appl. No.: 10/569,795

(22) PCT Filed: Aug. 4, 2004
(86) PCT No.: PCT/EP04/08747

§ 371 Date: Feb. 27, 2006

(30) Foreign Application Priority Data
Aug. 27, 2003 (FR) 0310224

(51) Int. Cl. C12Q 1/68; C07H 21/04; C12P 21/06; C12N 1/21; C07K 14/47

(52) U.S. Cl. 435/6; 435/69.1; 435/252.3; 435/320.1; 435/325; 530/350; 536/23.5

(57) ABSTRACT

The invention relates to an isolated human nucleic acid, characterized in that it corresponds to the promoter of the human FATP5 protein, and to an isolated human nucleic acid coding for a protein, particularly for the human FATP5 protein, placed under the dependence of said promoter. The invention further relates to a vector comprising said nucleic acid, or to a host cell into which said nucleic acid or said vector has been introduced. Finally, the invention relates to the use of said nucleic acid, said vector or said host cell in a method of identifying a product capable of modulating the expression of a nucieotide sequence placed under the dependence of said promoter of the human FATP5 protein, and to said method of identification. The invention finally relates to the product capable of modulating the expression of a nucleotide sequence placed under the dependence of said promoter of the human FATP5 protein, and to the use of said product in the preparation of a drug for preventing and/or treating diabetes or for inducing the β oxidation of fatty acids in the liver.

The present application relates to an isolated human nucleic acid, characterized in that it corresponds to the promoter of the human FATP5 protein, and to an isolated human nucleic acid coding for a protein, particularly for the human FATP5 protein, placed under the dependence of said promoter.

The invention further relates to a vector comprising said nucleic acid, or to a host cell into which said nucleic acid or said vector has been introduced. Finally, the invention relates to the use of said nucleic acid, said vector or said host cell in a method of identifying a product capable of modulating the expression of a nucleotide sequence placed under the dependence of said promoter of the human FATP5 protein, and to said method of identification.

The invention finally relates to the product capable of modulating the expression of a nucleotide sequence placed under the dependence of said promoter of the human FATP5 protein, and to the use of said product in the preparation of a drug for preventing and/or treating diabetes or for inducing the β-oxidation of fatty acids in the liver.

Type II diabetes is a major public health problem throughout the world. At present about 125 million people are affected by this disease, of which 15 million are in the USA alone. Predictions show that in 2010 the number of patients affected by diabetes will have doubled. Between 90 and 95% of people currently suffering from diabetes in the USA are affected by type II diabetes. Type II is a metabolic disease characterized by insulin resistance and hyperglycaemia and often associated with hypertension, lipid disturbance and obesity. In adults this pathological condition generally appears from the age of 30 and is characterized by complications including damage to the heart, blood vessels, eyes, kidneys and/or nerves.

As yet, unfortunately, no curative and/or preventive treatment for the causes exists, the treatments generally tending to address the consequences.

There is therefore a real need for products capable of treating the causes of diabetes. In order to identify said products capable of treating the causes of diabetes, there is also an ever-constant need for effective methods of identifying said products.

One of the objects of the present invention is to provide such a method.

Long chain fatty acids (LCFA) are an important source of energy for the organism, providing ATP through their beta-oxidation in the mitochondrion. They are also substrates for a large number of diverse cellular processes such as the cellular signalling pathways or the regulation of gene expression. By way of example, fatty acids are essential for providing the heart with a constant and effective supply of energy.

LCFA penetrate the cells of the mucosa after lipolysis by the lipoprotein lipase, and are then esterified to triglycerides in the endoplasmic reticulum. The triglycerides are then integrated into lipoparticles complexed with apolipoproteins to form very low density lipoproteins (VLDL), or chylomicrons, and leave the cell by exocytosis to be discarded into the systemic circulation. The lipoprotein lipase transforms these particles, on the surface of the endothelial cells, into non-esterified fatty acids, which can subsequently bind to albumin.

The kinetics of the capture of fatty acids in rat cells break down into a rapid linear phase over the first 30 seconds, followed by a transition phase with a decrease in the initial capture rate, and a late period with a slow accumulation rate. The initial capture rate corresponds to a vectorial unidirectional flow of fatty acids and can be characterized by a calculated function of the fatty acid concentration applied to the incubation medium. The kinetics, the competition by LCFA and the inhibition of fatty acid transport by treatment of the cell with a protease argue in favour of a process mediated by a transporter.

The first fatty acid transport protein (FATP) was identified in 1994 in rat adipocytes. Two corresponding genes were then identified and are now called FATP1 and acyl-coenzyme A synthetase (ACS). Screenings of mouse gene libraries have subsequently revealed a family of proteins characterized by a signature sequence consisting of 311 highly conserved amino acids in all the members of the FATP family. These all contain an AMP binding site. 6 mouse FATPs and 6 human FATPs have so far been identified. Coenzyme A synthetase activity is now associated with FATP1, 2, 4 and 5. Their role in LCFA capture can be deduced from their enzymatic activity. Alternatively, the expression of FATP can lead to an increase in the intracellular level of free fatty acids and hence can induce a VLACS (very long chain acyl-CoA synthetase) activity. In fact, the enzymes involved in acylation, for example acyl-coenzyme A synthetase (ACS), are positively regulated by PPAR (peroxisome proliferator-activated receptor) transcription factors and certain long chain fatty acids.

Acylation activates fatty acids to form active metabolic derivatives of acyl-CoA, which become capable of entering any metabolic pathway. In the endoplasmic reticulum, fatty acids can be involved in the synthesis of triglycerides. In the mitochondrion and in peroxisomes, FATPs can lead to the degradation of fatty acids.

FATPs have different subcellular locations depending on their amino termini. Their N-terminal ends could interact with different partners so as to distribute the fatty acids into different subcellular compartments and then induce their incorporation into separate metabolic pathways.

Although they possess at least two homologous signature units, the different FATPs have a separate chromosomal location, specific promoters and gene regulation, a specific distribution profile, a different cellular location and specialized functions.

In the prior art, FATP5 is found under different names, such as very long chain acyl-CoA synthetase relative (VLACSR), very long chain acyl-CoA synthetase homologue (VLCS-H2), cholyl-coenzyme A ligase or bile acid coenzyme A synthetase (BACS), depending on the activities detectable for this protein.

In humans the protein is expressed exclusively in the liver.

In mice it is known that a 2.6 kb messenger RNA is highly abundant in the liver. Lower levels of expression of shorter messenger RNAs have been detected in the brain, lungs, testicles and spleen (2.5 kb) and in the skeletal muscle (2.2 kb). The presence of transcripts can be demonstrated in the heart, but not in the kidney, by the PCR technique.

In humans, no transcript has been detectable in the fibroblast, brain or heart, in contrast to its murine homologue.

Southern blot analyses show that the VLACSR gene is present in a single copy in both humans and mice.

It has been reported, for example, that FATP5 is capable of activating long chain fatty acids and very long chain fatty acids (for example C18 to C26). FATP5 is capable of activating chenodeoxycholate, deoxycholate, lithocholate and trihydroxycholestanoic acid.

To the inventors' knowledge, the involvement of FATP5 in fatty acid degradation has never been. described. The role of FATP5 might be limited only to the reactivation of fatty acids through enterohepatic recycling.

In the prior art, international patent application WO 01/21795 suggests that FATP5 plays a role as fatty acid transporter, thus playing a role in the process of fatty acid capture by the cell.

Furthermore, it is known that people affected by diabetes or people who are liable to develop diabetes have a high level of free fatty acids in the bloodstream. An excess of free fatty acids in the plasma acts like a competitor for the utilization of glucose to produce energy in insulin-sensitive tissues, for example skeletal muscle. Fatty acids interfere at different levels in the glucose metabolism. They indirectly inhibit glycolysis, reduce the storage of glucose in the form of glycogen and indirectly inhibit glucose transport. This tends to show a preferential utilization of free fatty acids when they are in excess, and an increase in the concentration of glucose in the circulation, leading to the development of hyperglycaemia.

Surprisingly and unexpectedly, the inventors have now discovered that FATP5 plays a role in the degradation of fatty acids in the liver. Likewise, they have been able to show that the consequence of this property is significantly to reduce the glucose and lipid concentration in the plasma. In particular, they have been able to show that the level of expression of the messenger RNAs of FATP5 is greatly reduced in diabetic fatty rats (Zucker diabetic fatty rats (ZDF rats)) compared with the level of expression of the messenger RNAs of FATP5 in non-diabetic rats (Zucker lean rats (ZLC rats)).

It is thus possible to imagine that the consequence of an overexpression of FATP5 might be to reduce the level of free fatty acids, so one may well envisage that any product having the ability to increase the level of expression of FATP5 might be a good candidate for the preparation of a drug for the preventive or curative treatment of diabetes.

The present invention falls within this field of application.

The inventors have isolated a human nucleic acid sequence, characterized in that it comprises at least the nucleotide sequence identified under the number SEQ ID no. 1 in the annexed sequence listing, as being the promoter of the human FATP5 gene. This sequence is only slightly homologous with the sequence considered in the prior art as being that of the FATP5 promoter.

The invention thus relates primarily to an isolated human nucleic acid sequence, characterized in that it comprises at least the nucleotide sequence identified under the number SEQ ID no. 1 in the annexed sequence listing. Said sequence corresponds to the promoter of the human FATP5 gene.

The invention further relates to an isolated human nucleic acid sequence, characterized in that it consists of the nucleotide sequence identified under the number SEQ ID no. 1 in the annexed sequence listing.

In one particular embodiment of the invention, the nucleic acid sequence comprising at least the nucleotide sequence identified under the number SEQ ID no. 1 codes for a protein, particularly the human FATP5 protein. Particularly preferably, said nucleic acid sequence comprising at least the nucleotide sequence identified under the number SEQ ID no. 1, and coding for the human FATP5 protein, corresponds to the nucleotide sequence identified under the number SEQ ID no. 2 in the annexed sequence listing.

The invention further relates to the sense or antisense nucleic acid sequences complementary to the above, and to any nucleic acid sequence having a percentage identity of at least 80%, preferably of at least 90%, with one of the nucleic acid sequences according to the invention.

A nucleic acid sequence having a percentage identity of at least X % with a reference sequence is defined in the present invention as a nucleic acid sequence which can include up to 100-X alterations per 100 nucleotides of the reference sequence, while conserving the functional properties of said reference sequence. In terms of the present invention, “alterations” include consecutive or interspersed deletions, substitutions or insertions of nucleotides in the reference sequence.

A nucleic acid sequence having a percentage identity of at least 80%, preferably at least 90%, according to the invention includes all sequences that correspond to allelic variants, i.e. to individual variations of the sequences SEQ ID no. 1 or SEQ ID no. 2. These natural variant sequences correspond to polymorphisms present in mammals, particularly in humans.

The invention further relates to a cloning and/or expression vector into which the nucleic acid sequence of the invention as defined above is inserted. Preferably, the vector of the invention contains a nucleic acid sequence comprising at least the nucleotide sequence identified under the number SEQ ID no. 1 in the annexed sequence listing.

In one particular embodiment of the invention, the vector of the invention also comprises a nucleic acid sequence coding for a detectable protein placed under the dependence of the nucleic acid sequence identified as the promoter of the human FATP5 gene as defined above. In particular, this promoter corresponds to the sequence SEQ ID no. 1. “Detectable protein” is understood as meaning any protein which, once expressed, may easily be identified by any technique known to those skilled in the art. FATP5 itself, or proteins used as markers, for example green fluorescent protein (GFP), renilla or luciferase, may be mentioned among the proteins which can be used.

Such a vector can contain the elements necessary for the expression and optionally the secretion of the protein in a host cell.

Said vectors preferably contain translation initiation and termination signals as well as appropriate transcription regulation regions. They must be able to be stably maintained in the cell and can optionally comprise sequences coding for particular signals specifying the secretion of the translated protein.

The nucleic acid sequence according to the invention can be inserted into autonomous replication vectors within the chosen host or into integrating vectors of the chosen host.

Among the autonomous replication systems, it is preferable to use systems of the plasmid or viral type, according to the host cell.

The plasmid vectors which can be used according to the invention can be any known plasmids that allow the expression of a nucleic acid sequence. “Expression of a nucleic acid sequence” is understood according to the invention as meaning the ability of the vector of the invention to allow the transcription of the nucleic acid sequence of the invention into RNA, optionally followed by the translation of said RNA into protein.

Preferably, the plasmid according to the invention allows the expression of the isolated human nucleic acid sequence as described above. Particularly preferably, the plasmid according to the invention allows the expression of a nucleic acid sequence, characterized in that it comprises at least the nucleotide sequence identified under the number SEQ ID no. 1 in the annexed sequence listing, especially a nucleic acid coding for FATP5.

The following may be mentioned as examples of plasmids which can be used according to the invention: plasmids pCMVTag (Stratagene, La Jolla, USA), pcDNA3 (Invitrogen, Cergy Pontoise, France), pSG5 (Stratagene, La Jolla, USA) or pGL2 and pGL3 (Promega, Mannheim, Germany).

The viral vectors can be especially adenoviruses, retroviruses, lentiviruses, pox viruses or herpes viruses. Those skilled in the art are familiar with the technologies which can be used for each of these systems.

If it is desired to integrate the nucleic acid sequence into the chromosomes of the host cell, it is possible to use systems of the plasmid or viral type, for example; such viruses are e.g. retroviruses or adeno-associated viruses (AAV).

Among the non-viral vectors, preference is given to naked poly-nucleotides such as naked DNA or RNA, bacterial artificial chromosomes (BAC), yeast artificial chromosomes (YAC) for expression in yeast, mouse artificial chromosomes (MAC) for expression in murine cells and, preferably, human artificial chromosomes (HAC) for expression in human cells.

According to the invention, the vector is preferably a plasmid or an adenovirus.

Such vectors are prepared by the methods commonly used by those skilled in the art, and the resulting recombinant vectors can be introduced into the appropriate host by standard methods, for example lipofection, electro-poration, thermal shock, transformation after chemical permeabilization of the membrane, or cellular fusion.

The invention further relates to the transformed host cells, especially the eukaryotic and prokaryotic cells, into which at least one nucleic acid sequence according to the invention or at least one vector according to the invention has been introduced.

Bacterial cells, yeast cells and animal cells, particularly mammalian cells, may be mentioned among the cells which can be used in terms of the present invention. Insect cells, in which methods involving e.g. baculoviruses can be used, may also be mentioned.

The invention further relates to a method of producing a protein, characterized in that a host cell as described above is cultivated under conditions that allow the expression, in the form of a protein, of the nucleic acid sequence according to the invention which has been introduced into said cell.

The invention further relates to the use of the nucleic acid sequence of the invention, a vector or a host cell, as described above, for the identification of a product capable of modulating the expression of a nucleotide sequence placed under its dependence.

The invention further relates to a method of identifying a product capable of modulating the expression of a nucleotide sequence placed under the dependence of the nucleic acid sequence according to the invention as described above, characterized in that

a) a nucleic acid sequence, a vector or a host cell, as described above, is brought into contact, in a medium suitable for the expression of a nucleotide sequence, with a product capable of modulating the expression of a nucleotide sequence placed under the dependence of the nucleic acid sequence as described above; and

b) the level of expression of said nucleotide sequence placed under the dependence of the nucleic acid sequence as described above is measured.

“Medium suitable for the expression of a nucleotide sequence” is understood here as meaning any expression system that allows the synthesis of an RNA, particularly a messenger RNA, and optionally a protein from the nucleic acid sequence of the invention.

In one particular embodiment, the method of the invention comprises an additional step for comparison of the level of expression determined in b) with the level of expression of said nucleotide sequence placed under the dependence of the nucleic acid sequence as described above, in a control which has not been brought into contact with the product to be identified.

The comparison makes it possible to evaluate the modulating ability of the test product in respect of the nucleic acid sequence of the invention.

“Modulate” or “modulating ability” is understood according to the invention as meaning the ability of the test product to stimulate or inhibit the expression of a nucleotide sequence placed under the dependence of the nucleic acid sequence according to the invention.

The level of expression of the nucleotide sequence placed under the dependence of the nucleic acid sequence of the invention can be measured by the conventional techniques of mRNA or protein analysis which are known per se; the following techniques may be mentioned as non-limiting examples: RT-PCR, Northern blofting, Western blotting, RIA, ELISA, immunoprecipitation, and immunocytochemical or immunohistochemical analysis techniques. For everything relating to the experimental protocols in cellular or molecular biology or immunocytochemical or immunohistochemical analysis, reference may be made here and elsewhere in the present patent application to the numerous works thoroughly familiar to those skilled in the art, particularly “Current Protocols in Immunology; John Wiley and Sons, Teton Data System, Jackson, Wyoming (ISBN 0-471-30660-6, 2003)”.

Advantageously, said measurement is made with the aid of probes, primers or antibodies.

The products capable of modulating the expression of a nucleotide sequence placed under the dependence of the nucleic acid sequence according to the invention can be biological macromolecules such as a nucleic acid, a lipid, a sugar, a protein, a peptide, a protein-lipid, protein-sugar, peptide-lipid or peptide-sugar hybrid compound, or a protein or peptide to which chemical branchings or chemical molecules have been added.

The invention further relates to a product capable of modulating the expression of a nucleotide sequence placed under the dependence of the nucleic acid sequence according to the invention, said product being obtainable by the method of the invention.

The invention further relates to a method of identifying a product capable of interacting with the nucleic acid sequence according to the invention, characterized in that

a) a nucleic acid sequence, a vector or a host cell, as described above, is brought into contact with a product capable of interacting with the nucleic acid sequence according to the invention; and

b) the interaction between said nucleic acid sequence and said test product is evaluated.

In one particular mode of carrying out the invention, the purpose of said method of identification is to identify a product capable of binding to the nucleic acid sequence according to the invention, the method being, for instance, calorimetry in the case of heterologous interactions or the double hybrid test in the case of peptide-peptide interactions.

Finally, the invention relates to a product capable of interacting with or binding to the nucleic acid sequence according to the invention, said product being obtainable by one of the methods of identification as described above.

According to the invention, the interaction and/or the binding between said nucleic acid sequence and said test product can be evaluated by any known technique.

The inventors have shown that FATP5 plays a role in the degradation of fatty acids in the liver. Likewise, they have been able to show that a consequence of this property is significantly to reduce the plasma glucose and lipid concentration. In particular, they have been able to show that the level of expression of the FATP5 messenger RNAs is greatly reduced in diabetic rats (Zucker diabetic fatty rats (ZDF rats)) compared with the expression of the FATP5 messenger RNAs in non-diabetic rats (ZLC rats).

Thus the invention further relates to the use of the product according to the invention in the preparation of a drug for preventing and/or treating diabetes.

The invention further relates to the use of the product according to the invention in the preparation of a drug for inducing the beta-oxidation of fatty acids in the liver.

In addition to the foregoing provisions, the invention also includes other provisions which will become apparent from the following description referring to Examples of how to carry out the invention and to the attached drawings, in which:

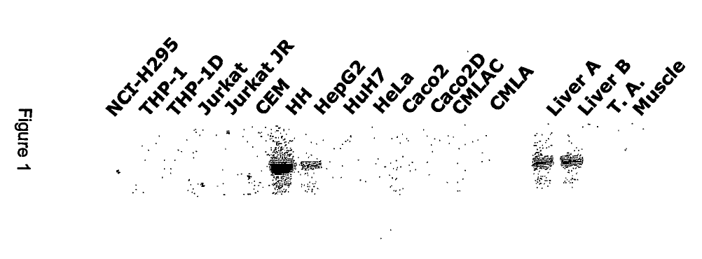

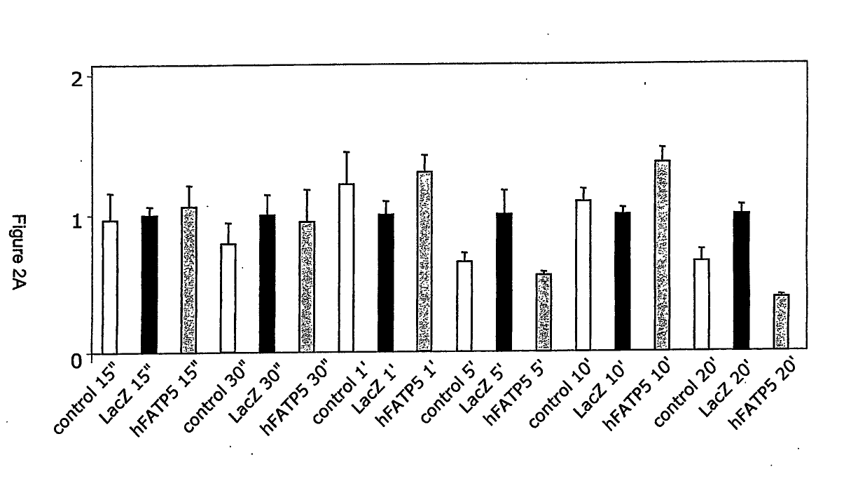

FIG. 1 shows the cell and tissue distribution of the expression of the human FATP5 gene, analysed by PCR. NCl-H295: adrenal cell line; THP-1: monocyte; THP1D: differentiated macrophage of THP-1 monocyte; Jurkat and Jurkat J R: T cells; CEM: cell of acute lymphoblastoid leukaemia cell line; HH: human primary hepatocytes; HepG2, HuH7: human hepatic cells; CaCo2: colon cells; CaCo2D: differentiated CaCo2 cells; CASMC: coronary artery smooth muscle cells; ASMC: aorta smooth muscle cells.

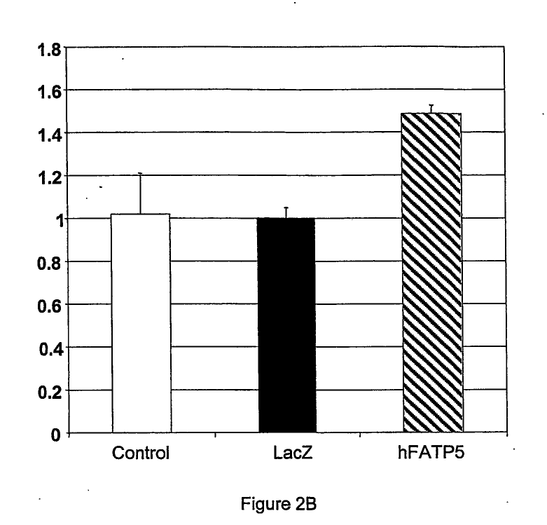

FIG. 2 shows the results obtained in fatty acid capture tests:

A: test on 3T3-L1 cells;

B: test on rat hepatocytes (RH) (time: 1 minute).

The results represent the induction of fatty acid capture as a function of time for each condition. The control condition represents the measurement of capture by cells infected with an adenovirus carrying a gene whose expression has no influence on fatty acid capture by the cell.

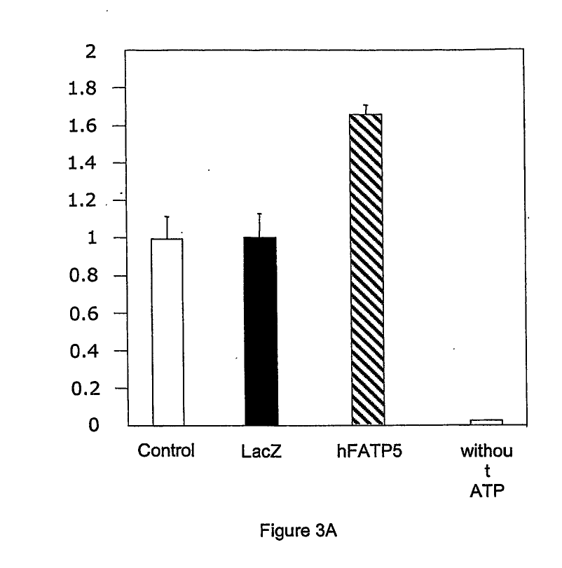

FIG. 3 shows the results obtained in tests for determining the ACS activity (FIG. 3A) and the VLACS activity (FIG. 3B) after infection with the adenovirus carrying the hFATP5 gene. The results are expressed as the induction factor.

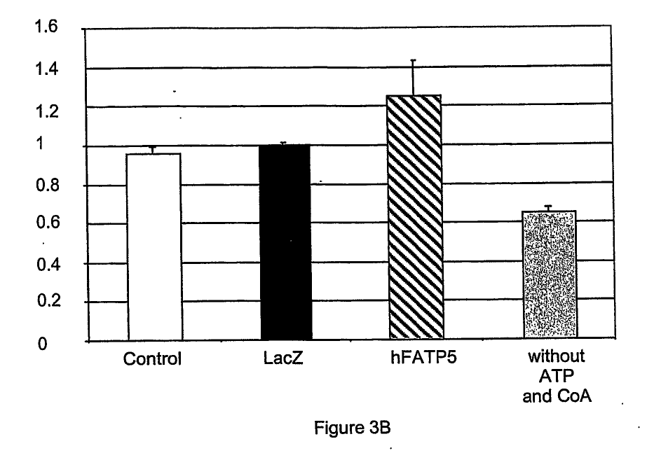

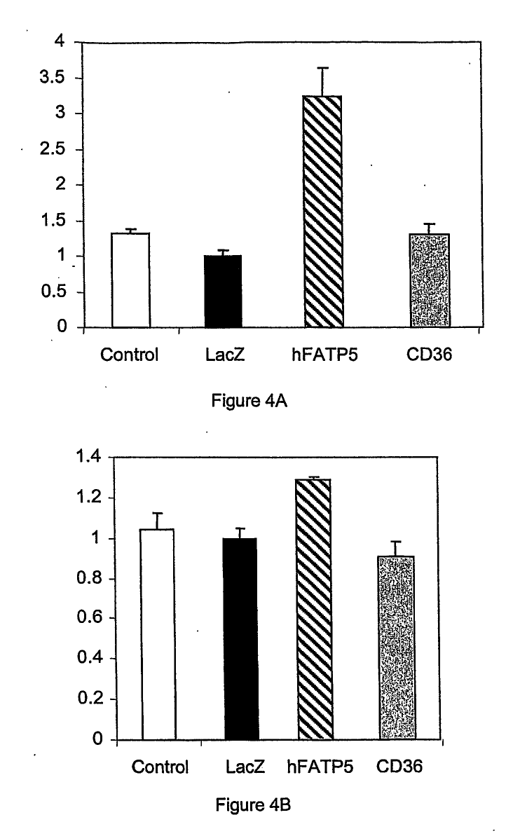

FIG. 4 shows the results obtained in tests for determining the effect of the expression of hFATP5 on the β-oxidation of fatty acids:

4A: HepG2 at 48 hours;

4B: RH at 12 hours.

The results are expressed as the induction factor.

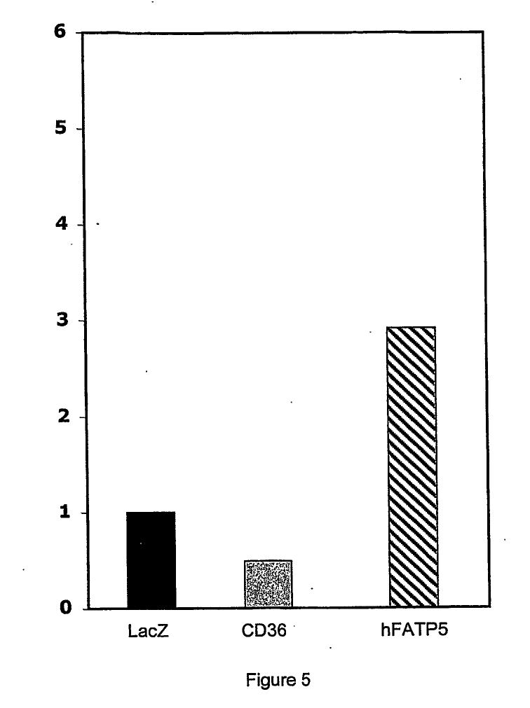

FIG. 5 shows the results obtained in tests for determining the effect of the expression of hFATP5 on the degree of esterification of fatty acids in HepG2 cells at 48 hours. The results are expressed as the 3H2O/3TG ratio.

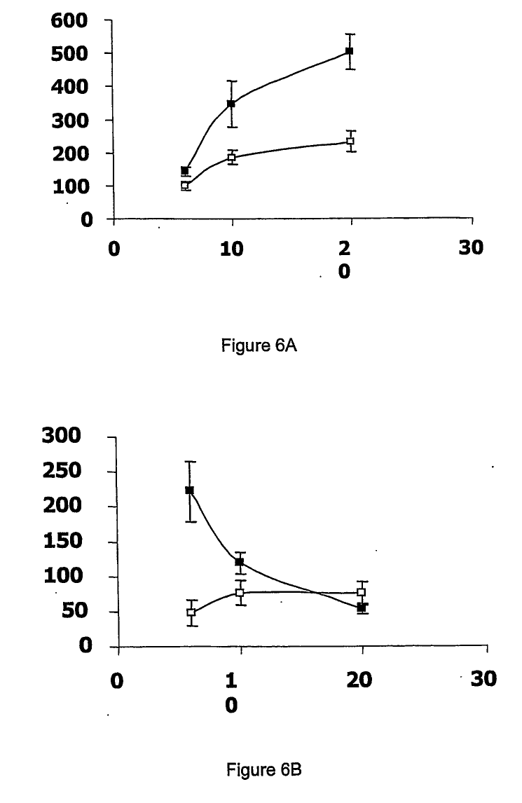

FIG. 6 shows the results obtained in tests for determining the effect of the expression of hFATP5 on regulation of the expression of hFATP5 during the development of diabetes in Zucker diabetic fatty rats:

A: measurement of the glucose level;

B: measurement of the insulin level;

-▪-: male ZDF rats (fa/fa);

-□-: male ZLC rats (fa/+).

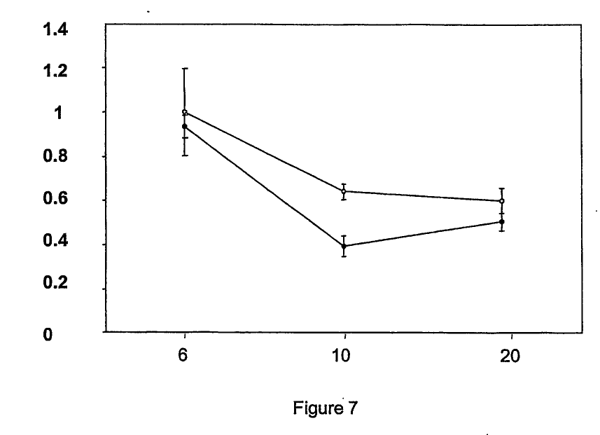

FIG. 7 shows the results obtained in tests for determining the level of hFATP5 messenger RNAs in Zucker diabetic fatty rats (ZDF: -λ-) and in ZLC rats (-Γ-).

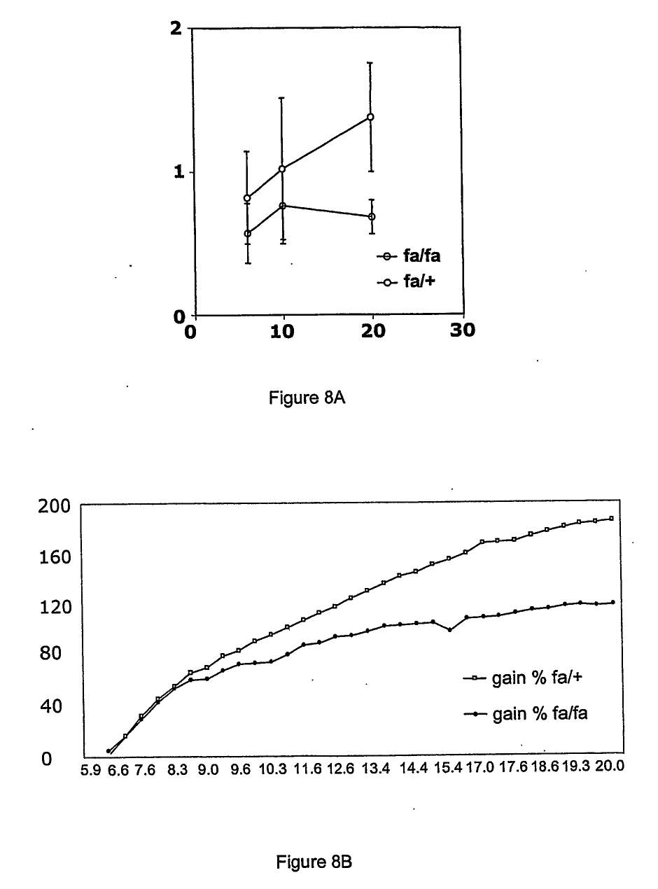

FIG. 8 shows the results of the biochemical analyses:

A: measurement of the free fatty acid level in the liver (-θ-: ZDF rats (fa/fa); -o-: ZLC rats (fa/+));

B: weight gain curves (-λ-: ZDF rats (fa/fa); -□-: ZLC rats (fat+)).

FIG. 9 shows the degree of acylation of oleates in 8-week-old ZDF and ZLC rats at 5 and 30 minutes for each condition. A: ZDF rats; B: ZLC rats.

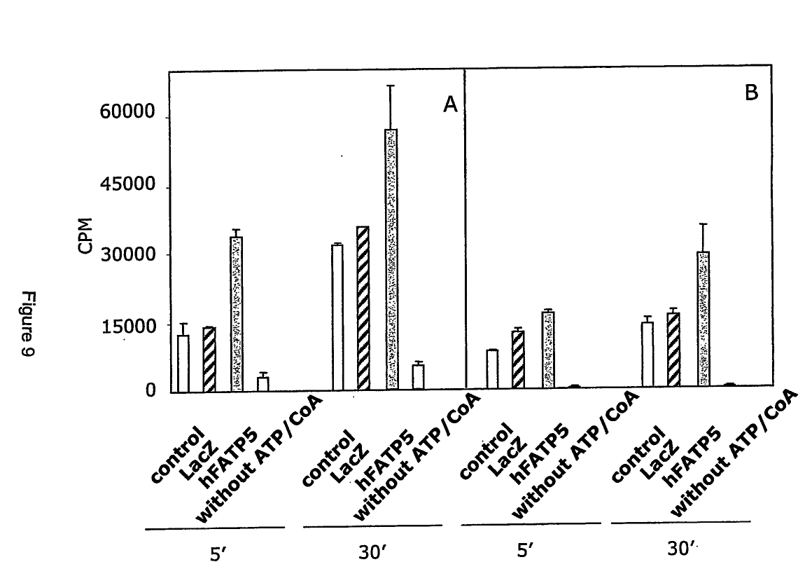

FIG. 10 shows the reversal of inhibition of the degrees of beta-oxidation in the primary hepatocytes of ZDF rats compared with ZLC control rats. A: 8-week-old rats; B: 10-week-old rats. (C=control; Z=LacZ; h5=hFATP5).

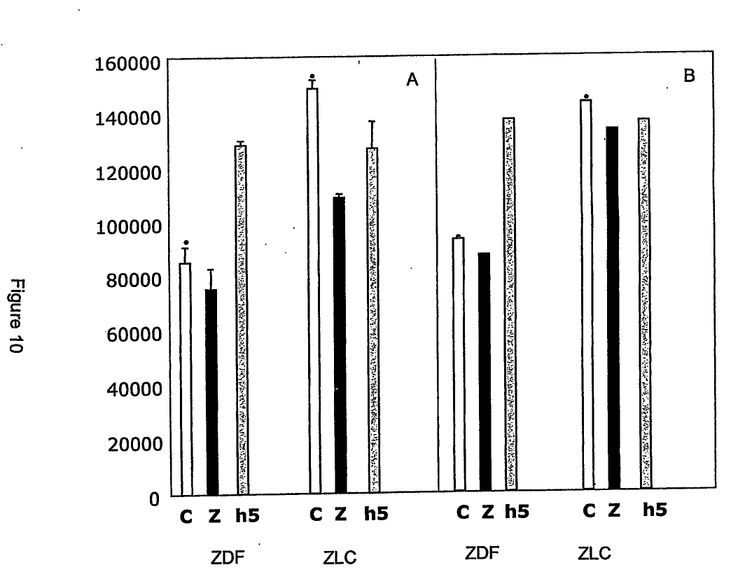

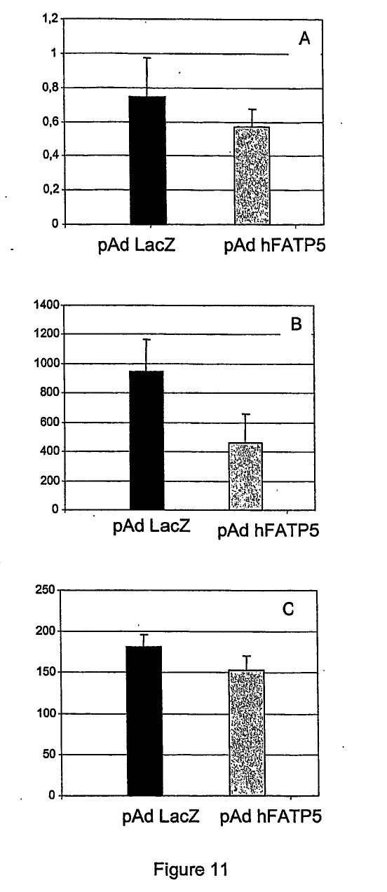

FIG. 11 shows the measurement of biochemical parameters in ZDF rats infected with the adenovirus carrying FATP5, compared with rats infected with the LacZ control adenovirus (free fatty acids (A), triglycerides (B) and glucose (C)).

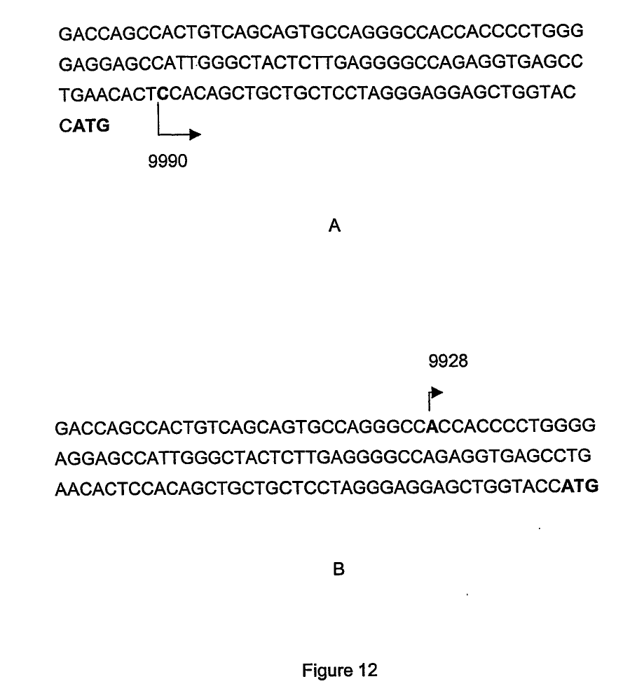

FIG. 12 shows the location of the transcription start site (TSS) of the FATP5 gene. A: predictive methodology by bioinformatics; B: experimental methodology by RACE.

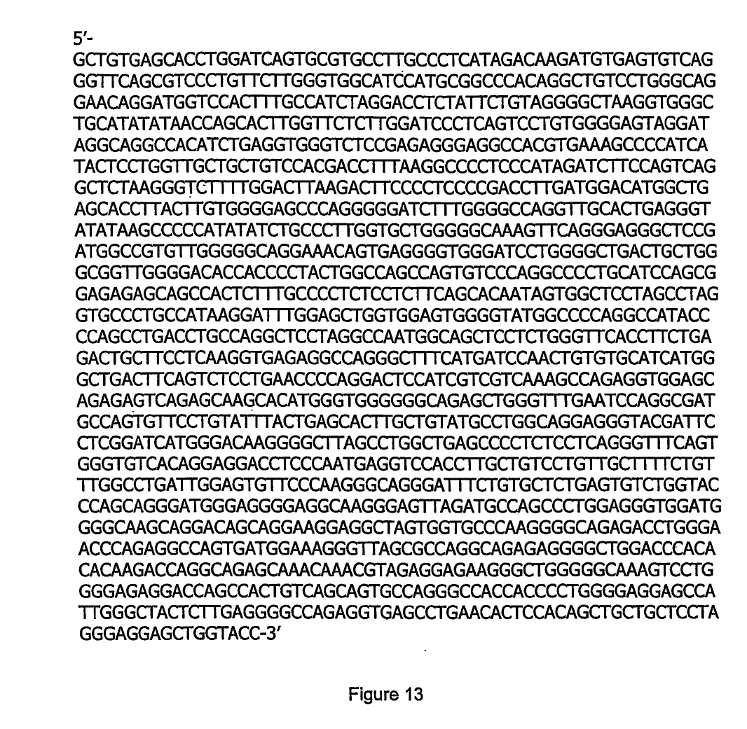

FIG. 13 shows the sequence of the putative promoter of the human FATP5 gene. This sequence is identified under the number SEQ ID no. 1 in the annexed sequence listing.

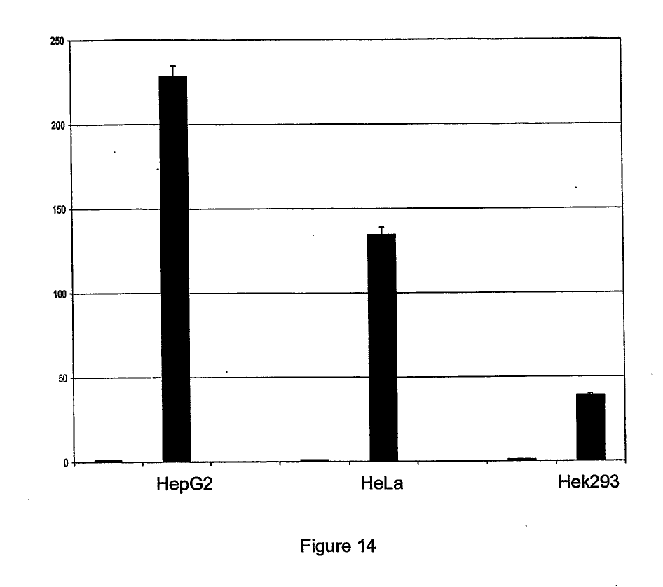

FIG. 14 shows the activity of the promoter sequence of the human FATP5 gene in HepG2, HeLa and Hek293 cells. The results are presented as the induction relative to the control measured on cells transformed with void plasmid pGL3.

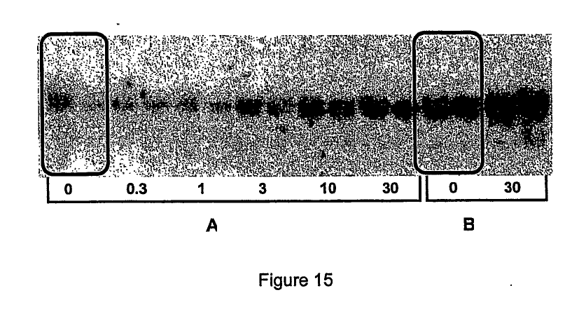

FIG. 15 shows the variation in the level of FATP5 messenger RNAs after treating ZDF rats (A) or ZLC rats (B) with a compound known to be an insulin sensitizer.

The Examples which follow illustrate the invention without in any way implying a limitation.

EXAMPLE 1

### Demonstration of the Involvement of hFATP5 in the β-Oxidation of Fafty Acids

Materials and methods

A) FATP5 expression profile

a) Preparation of the RNA

Total RNA is prepared from cells and tissues using the technique described by Chomczynski and Sacchi (Chomczynski et al., 1987). The RNAs are quantified by measurement of the optical density at 260 nm and the quality of the RNAs is checked by measurement of the 260/280 optical density ratio.

1 μg of each sample is used as template in a reverse transcription (RT) reaction using the enzyme MMLV-RT (Gibco BRL, Paisley, UK) and the polymerase chain reaction is performed on 2 μl of the RT product. Specific primers (Eurogentec, Seraing, Belgium) were prepared for hFATP5 and rFATP5. To assure the specificity of the PCR products, the thermal denaturation temperature (Tm) is optimized for each specific target and the number of cycles is situated between 25 and 30, depending on the abundance of the transcription product.

TABLE I
Description of the primers

| Identity | | | |
|---|---|---|---|
| of primer | Sequence | Tm: ° C. | Cycles |
| Actin r | GACTACCTCATGAAGATCCTGACTGA | 55 | 20-25 |
| sense | GCG | | |
| Actin r | GGGGCAATGATCTTGATCTTCATGGT | | |
| antisense | | | |
| GAPDH h | CCACATACCAGGAAATGAGC | 55 | 20-25 |
| sense | | | |
| GAPDH h | GACATCAAGAAGGTGGTGAA | | |
| antisense | | | |
| FATP5 h | GCTCTAGAGCTGGTACCATGGGTGTC | 60 | 25 |
| sense | AGGCAACAG | | |
| FATP5 r | GGGGTACCAATGCCATGGGTATTTGG | | |
| sense | AAGAAACTAACC | | |
| FATP5 r | GGGAACTGGACTTCGGGCAAATGTGT | | |
| antisense | GG | | |

The DNA PCR products are analysed on a 1% agarose gel. The signal is quantified as densitometric values using a Gel Doc™ 2000 instrument (Bio-rad, Marnes La Coquette, France). For the in vivo transcriptional analysis, the quantification is calculated as the ratio of the values of each specific target to the value of the internal control (actin). The results are expressed as induction levels relative to the control, which is set at 1.

The quantitative analysis is performed on a 32-capillary Light Cycler apparatus (Roche) with Sybergreen, which is a fluorophore labelling the double-stranded DNA, by means of a two-step PCR (Roche, Mannheim, Germany). The results for each target, formulated as the crossing points of the differential of the amplification curve (representing the increase in fluorescence during the PCR) on the ordinate with the number of cycles on the abscissa, are normalized with a control gene.

TABLE II
Conditions of the quantitative PCR

| | | Tm of final |
|---|---|---|
| Insert | Programme | product (° C.) |
| RFATP5 | 8′, 95° C., 40 cycles (15″, 94° C.; 10″, | 83 |
| | 55° C.; 15″, 72° C.) | |

B) Kinetics of the Development of Diabetes in Zucker Diabetic Fatty Rats

5-week-old male Zucker diabetic fatty rats (ZDF/GMI-fa/fa) and male Zucker lean rats of the same age, originating from Genetic Model Inc. (Charles River Laboratories, Wilmington, Mass., USA), are fed with unrestricted amounts of Purina 5008 (PMI Nutrition International, Wellingborough, Northamptonshire, UK).

At the ages of 6 weeks, 10 weeks and 20 weeks, 48 rats are divided into 3 groups of 16 animals (8 Zucker diabetic fatty rats and 8 Zucker lean rats). The weight of the animals and their food consumption are monitored throughout the experiment.

Blood samples are collected by retro-orbital puncture under anaesthesia after a diet period of 8 hours during the day. The anaesthesia is induced by the intraperitoneal injection of pentobarbital. The animals are sacrificed by cervical dislocation and the liver is removed, weighed and immediately frozen in liquid nitrogen for additional analyses.

a) Table of Study Progression

| Week | | Age of animals |
|---|---|---|
| 0 | Reception of 24 ZDF and 24 ZLC | 5 weeks |
| 1 | Sacrifice of group 1 (8 ZDF/8 ZLC) | 6 weeks |
| 5 | Sacrifice of group 2 (8 ZDF/8 ZLC) | 10 weeks |
| 15 | Sacrifice of group 3 (8 ZDF/8 ZLC) | 20 weeks |

b) Analyses

The serum glucose, the cholesterol, the triglycerides and the free fatty acids are determined using enzymatic biological assays.

The insulin levels are determined using radioimmunological analyses.

Part of the liver is kept for histological studies.

C) Cloning of the Complete Sequence of hFATP5 Into Adenoviruses

Unless indicated otherwise, the techniques used are those described by the suppliers or in the numerous classical works on molecular biology.

The complete coding sequence of hFATP5 (from the ATG in position 1575 of the sequence SEQ ID no. 2) is cloned into the expression system PT3414-1 of Adeno-X (Clontech, Palo Alto, USA).

The total RNAs of a human hepatocyte culture are extracted with the RNeasy miniprep kit from Qiagen (Qiagen, Courtaboeuf, France).

A first DNA strand is then synthesized as follows:

| RNA | qsp 1 | μg |
|---|---|---|
| RNAs inhibitor (Promega, Charbonnières, France) | 1 | μl |
| First strand buffer 5× (Invitrogen) | 6 | μl |
| DTT (10 mM) (Invitrogen) | 3 | μl |
| dNTP (10 mM) O (Promega, Charbonnières, France) | 0.375 | μl |
| pdN6 (0.2 μg/μl) (Amersham) | 1 | μl |
| H₂O | qsp 30 | μl |
| MMLV-RT (Invitrogen) | 1 | μl |
| Programme: | | |
| 20° C. | 15 | minutes |
| 37° C. | 60 | minutes |
| 95° C. | 5 | minutes |

A PCR is then performed with the aid of the following primers:

Sequence h5_4 As (Invitrogen, Cergy Pontoise,
France): SEQ ID no. 19:
GCTCTAGATCAGAGCCTCCAGGTTCCCTCACACACAGCC
Sequence h5_1S (Invitrogen, Cergy Pontoise,
France): SEQ ID no. 20:
GCTCTAGATGGTACCATGGGTGTCAGGCAACAG

using the following reaction medium:

| | |
|---|---|
| Oligo sense (10 pM) | 1 μl |
| Oligo antisense (10 pM) | 1 μl |

Taq platinum Pfx+buffer 10X+MgSO₄ (Invitrogen, Cergy Pontoise, France)

dNTP (Promega, Williamsburg, Iowa) in a final volume of 20 μl.

Amplification is then performed according to the following programme:

1 cycle at 94° C. for 5 minutes, followed by 25 cycles comprising 30 seconds at 94° C., then 30 seconds at 55° C. and 2 minutes at 68° C.

The product is then kept at 4° C.

The PCR fragment obtained is purified using the QIAquick PCR purification kit (Qiagen, Courtaboeuf, France).

The purified fragment is then digested with Xbal (NEB, Beverly, Mass.) and cloned into vector pShuttle of the pAdenoX kit from Clontech, according to the supplier's recommendations.

The plasmid obtained is introduced into Escherichia coli bacteria of the DH5d type and the clones are selected by their kanamycin resistance. The plasmid DNA of the positive clones is then purified, checked by restriction analysis and sequenced. The expression of the protein is validated in vitro using the T7 expression system. The expression cassette of pShuttle is cleaved using PI-Sce/l-Ceul and ligated to Adeno-X™ Viral DNA. The ligation product is digested with Swal and introduced into E. coli. The ampicillin resistant clones are selected.

The recombinant adenoviral DNA containing the gene of interest is digested with Pacl and introduced into embryonic human kidney cells (293 cells). The adenoviral recombinants are harvested and reamplified by 3 amplification cycles. The virus is purified by the CICs method and then cleaned in a Sephadex G50 column. The viral particles are titrated by a plaque forming assay (Pfu) in 96-well plates.

D) Preparation of the Rat Hepatocytes

Rat hepatocytes are isolated from male rat livers by collagenase perfusion. The rats are anaesthetized by an intraperitoneal injection of pentobarbital. The livers are then perfused in the portal vein, firstly with 200 ml of liver perfusion medium and then with 200 ml of Hanks buffer supplemented with 10 mM Hepes, 4 mM CaCl₂ and 14 mg of Blendzyme3. The livers are dissected, chopped in Hepatocyte Wash medium (Gibco BRL, Paisley, UK) and filtered on 70 μm filters.

The cells are centrifuged and washed 3 times in Hepatocyte Wash buffer before the viability of the cell is estimated by the Trypan Blue exclusion technique (viability>85%). The cells are inoculated into Williams medium supplemented with UltroserSF (2% by volume), penicillin (100 U/ml), strepto-mycin (100 μg/ml), free fatty acids/BSA (SIGMA, St. Louis, Mo., USA) (0.2% by weight/volume), L-glutamine (2 mM), dexamnethasone (1 μM), triiodothyronine (T3, SIGMA, St. Louis, Mo., USA) (100 nM) and insulin (100 nM). After 4 hours, the culture medium is replaced with the same Williams medium without Ultroser or BSA.

E) Infection of ZDF Rats with the hFATP5 Adenovirus and the LACZ Adenovirus During the Development of Inverse Diabetes

5-week-old male Zucker diabetic fatty rats (ZDF/GMI-fa/fa) and male Zucker lean rats of the same age, originating from Genetic Model Inc. (Charles River Laboratories, Wilmington, Mass., USA), are fed with unrestricted amounts of Purina 5008 (PMI Nutrition International, Wellingborough, Northamptonshire, UK).

At the age of 8 weeks, 5.10⁹ Pfu of adenovirus or PBS (for the controls) are injected into a caudal vein of the rats. The volume injected is less than 2 ml per rat. 3 rats are infected with pAdLacZ and 3 with pAdhFATP5. The rats are sacrificed at 10 weeks. The glucose, free fatty acid and triglyceride levels are measured in the blood plasma.

F) Functional Tests

a) Capture of the fatty acids 20 μM final of C14-labelled oleic acid (FIG. 2B) or 78 nM final of tritium-labelled oleic acid (FIG. 2A) complexed with BSA are used in the transport tests.

Rat hepatocytes (10⁶cells/compartment) are inoculated into the wells of 6-well plates and incubated for 4 hours at 37° C. 3T3-L1 cells (ATCC no. CL-173, Manassas, Va., USA) (5.10⁵ cells/compartment) were incubated in DMEM containing 2% of foetal calf serum (FCS). The infection has a multiplicity of 1 and is continued overnight.

The cells are treated with trypsin, harvested, centrifuged and resuspended with PBS to give a suspension of 5.10⁵ cells/ml. 200 μl of cellular suspension (10⁵ cells) are placed in 5 ml polypropylene centrifuge tubes. The cellular suspensions in PBS are preincubated for 5 minutes at 37° C. in a water bath, with shaking. An equal volume of a stock solution of fatty acids/BSA, concentrated 2-fold, is added to each tube for capture of the fatty acids. At given intervals, the capture was stopped by adding to each tube 5 ml of a cold solution (0° C.) of PBS containing 0.1% of BSA and 200 μM phloretin (SIGMA, St. Louis, Mo., USA) (wash solution). The cells are centrifuged and washed twice. The centrifugation residue is resuspended with 400 μl of lysis buffer solution (0.4% SDS in water). 4 ml of scintillator solution are then added and the incorporated fatty acids are counted in a Tri-Carb 2100 counter (Packard, Meriden, Conn., USA). The results have been presented as a proportion of the induction of LacZ, normalized to 1.

b) Acylation of the Fatty Acids

Rat hepatocytes (10⁶ cells/compartment) are inoculated into the wells of 6-well plates and incubated for 4 hours at 37° C. The medium is then withdrawn and 2 ml of the infectious mixture (DMEM, 2% FCS) are added to each well. The infection has a multiplicity of 1 and is continued overnight. The cells are treated with trypsin, harvested and centrifuged. The centrifugation residue is resuspended in 300 μl of buffer solution A (500 mM tris HCl at pH 8.5, 1 mM MgCl₂, 100 mM NaCl, 1 mM ATP, 0.1% of TritonX 100). The cells are ultrasonicated and incubated for 30 minutes in ice. The protein levels are quantified by Bradford's method. 20 μl of the extract, containing 20 μg of proteins, are used per test and placed in a glass tube. 180 μl of the reaction mixture (50 mM tris HCl at pH 8.5, 150 μM coenzyme A, 300 μM DTT, 10 mM ATP, 10 mM MgCl₂, 0.1% of TritonX 100, 10 μM palmitic acid or lignoceric acid labelled with C14) are added to start the test. The reaction is stopped after 5 to 30 minutes by the addition of 800 μl of 1% perchloric acid.

To evaluate the levels of labelled oleyl-CoA, the reaction mixture is extracted with 2.25 ml of isopropyl/heptane/sulfuric acid (40/10/1). The extracts are mixed and the organic phase is withdrawn. Two successive extractions are performed. 4 ml of scintillator solution are added to 1 ml of aqueous phase and counted in a Tri-Carb 2100 TR counter. The radioactivity measured on control extracts of boiled proteins is subtracted from the corresponding test in order to determine the amount of acyl-CoA.

The results are presented as a proportion of the induction of LacZ, normalized to 1.

c) Oxidation of the Fatty Acids

Rat hepatocytes (6.10⁵ cells/compartment) are inoculated into the wells of 12-well plates and incubated for 4 hours at 37° C. The infection has a multiplicity of 100 and is continued overnight. The medium is then withdrawn and 500 μl of DMEM, supplemented with 64 nM oleic acid [9,10 ³H] and 2% of BSA, are added to each well. After 12 hours of incubation at 37° C., the medium is transferred to a microtube and the excess oleic acid [³H] is precipitated with 50 μl of 10% trichloroacetic acid and 50 μl of 20% BSA. The mixture is centrifuged at 12,400 rpm for 2 minutes. The supernatant is transferred to a microtube and 500 μl of water are added. Incubation is continued at 50° C. for 18 hours. After the addition of 4 ml of scintillator solution, the tritiated water is measured and counted in a Tri-Carb 2100 counter.

The results are presented as a proportion of the induction of LacZ, normalized to 1.

d) Esterification of the Fatty Acids

Although the oxidation of the fatty acids was measured in the medium, the triglycerides are extracted from the cells incubated with the labelled fatty acids. Rat hepatocytes are treated with trypsin and mixed in triplicate in one and the same tube. The cells are centrifuged at 18,500 rpm for 2 minutes. Each well is washed with a bicarbonate-based solution (Krebs-Ringer bicarbonate buffer: 1.2 mM KH₂PO₄, 26 mM NaHCO₃, 1.3 mM MgCl₂, 124 mM NaCl, 5 mM KCl, 10 mM glucose) and this buffer solution is used to resuspend the centrifugation residue.

The cells are centrifuged a second time and the supernatant is with-drawn. This step is repeated. The centrifugation residue is then resuspended in 100 μl of water and can be frozen in liquid nitrogen for additional analyses. The centrifugation residue is then left to stand at room temperature (25° C.). 500 μl of acetone are then added and the mixture is dried under vacuum with the aid of a concentrator. The residue is resuspended with 300 μl of a chloroform/methanol mixture (1/1) containing 5 μl of 50 nM triolein as substrate. After mixing, the whole is centrifuged to remove the supernatant. Two extractions are performed. The centrifugation residue is resuspended in 30 μl of a chloroform/methanol mixture (1/1) and deposited on a thin layer chromatography plate precoated with G60 silicone gel. Migration is carried out for one and a half hours with 200 ml of buffer solution (H-hexane/diethyl ether/methanol/acetic acid 90/20/2/3).

Results:

A) Cell and Tissue Distribution of hFATP5

A PCR study confirms the exclusive expression of human FATP5 in the liver, primary hepatocytes and cells of the HepG2 human cell line.

These results are shown in FIG. 1.

B) Functional characterization of FATP5

a) Construction of the Adenoviruses

To characterize the functions of FATP5 in the metabolism of fatty acids in the liver, an adenovirus carrying the hFATP5 gene (pAdeno-hFATP5), a control adenovirus pAdeno-LacZ and an adenovirus pAdeno-CD36 were designed, the last one as a positive control for fatty acid transport.

b) Cagture of the Fatty Acids

The capture of oleates is tested on 3T3-1 cells (FIG. 2A) and on rat hepatocytes (RH) (FIG. 2B) isolated from Wistar rats infected with the adenovirus.

The overexpression of FATP5 has no influence on the fatty acid transport activity in 3T3-L1 cells (FIG. 2A), but significantly increases the fatty acid transport in rat hepatocytes.

This result suggests that the fatty acid transport activity is effected differently according to the cell line. This may be due to the effective recruitment of different cofactors specific to a cell line. Earlier results suggest that FATP may act in tandem with partners like ACS or FAT.

The results presented here suggest that FATP5 acts differently on fatty acid transport according to the cells in which the protein is expressed.

Furthermore, the absence of an FATP5 effect in preadipocyte cell lines and the rapid increase in fatty acid transport in rat hepatocytes suggests that FATP5 has a specific function which influences fatty acid transport in hepatocytes, rather than being a simple ubiquitous transporter.

c) Tests for the ACS and VLACS Activity

The ACS test is performed with palmitate and lignocerate in primary rat hepatocytes infected with the adenovirus. A significant increase in the VLACS activity after incubation with lignocerate, due to the overexpression of hFATP5, is only detectable with an incubation period of 30 minutes (FIG. 3A). It is suggested that the induction of the ACS activity by the overexpression of hFATP5 might be an indirect effect.

The very long chain acyl-CoA synthetase activity is slightly increased, but is specific for hFATP5, given that the overexpression of CD36 has no influence on the acylation of lignocerate (FIG. 3B).

The previously published results on the VLACS activity of FATP proteins are thus confirmed. However, there is a specific difference in the induction kinetics. The results suggest that FATP5 has a broader spectrum of activity, given that it shows significant effects on C16 fatty acids such as palmitate, which represent a larger class of fatty acids than the very long chain fatty acids.

d) Effect of the Expression of hFATP5 on the β-Oxidation of Fatty Acids

In order to analyse whether the degree of degradation of fatty acids is modulated by the overexpression of hFATP5, the conversion of tritiated oleate to tritiated water, a final product of the β-oxidation, was measured.

The overexpression of the CD36 control gene does not affect the β-oxidation in HepG2 cells (FIG. 4A) or in rat primary hepatocytes (FIG. 4B), which is in agreement with the hypothesis that CD36 directs the esterification of fatty acids and not their degradation.

By contrast, an overexpression of hFATP5 considerably increases the degree of P-oxidation in HepG2 hepatocytes (more than 3-fold) (FIG. 4A).

The overexpression of hFATP5 induces the degradation of fatty acids in primary hepatocytes.

e) Confirmation of the Involvement of hFATP5 in the β-Oxidation of Fatty Acids Rather than their Esterification

In order to confirm that the expression of hFATP5 leads only to the degradation of fatty acids and does not influence their degree of esterification, the β-oxidation test is complemented with an esterification test in the same experiment on HepG2 cells.

The overexpression of hFATP5 does not affect the degree of esterification (FIG. 5).

This clearly demonstrates a novel functional activity of hFATP5 that consists solely in the degradation of fatty acids in the liver, whereas FAT/CD36 directs fatty acids towards the esterification process. hFATP5 is a β-oxidation activator.

f) Regulation of the Expression of hFATP5 During the Development of Diabetes in Zucker Diabetic Fatty Rats

A study of the level of expression of the FATP5 gene in Zucker diabetic fatty rats during the appearance of NIDDM in this model was conducted and the results were correlated with the variation in insulin and glucose levels.

Zucker diabetic fatty rats naturally develop diabetes with the same progression as in man (FIGS. 6A and 6B), with:

a state of hyperinsulinaemia and euglycaemia, which is observed for 6-week-old male ZDF (fa/fa) rats;

a state of hyperinsulinaemia and hyperglycaemia, which is observed for 10-week-old male ZDF (fa/fa) rats; and

a state of insulin deficiency and hyperglycaemia, which is observed for 20-week-old male ZDF rats.

A study by means of quantitative and semiquantitative analyses (Table III and FIG. 7) of the expression of hFATP5 shows that the level of hFATP5. messenger RNAs decreases at 10 weeks in ZDF rats. Over the same period, the glycogen levels are distinctly higher in ZDF rats than in ZLC rats (Table III).

TABLE III
Analysis of the glucose metabolism, glycogen level and
FATP5 mRNA level (regulation of the messenger RNA) at
6, 10 and 20 weeks in ZDF rats versus ZLC rats

| | 6 weeks | | 10 weeks | | 20 weeks | |
|---|---|---|---|---|---|---|
| | SQ analysis of the mRNA levels | | | | | |
| | FAT | FAT5 | FAT | FAT5 | FAT | FAT5 |
| ZDF/ZLC | 1.6 | 0.9 | 2.6 | 0.5 | 1.5 | 0.9 |
| SEM* | 0.09 | 0.04 | 0.37 | 0.07 | 0.11 | 0.08 |

| | | | |
|---|---|---|---|
| Histology | Glycogen: + | Glycogen: ++ | Glycogen: + |
| of the | | | |
| liver | | | |
| Insulin/ | Hyperinsulinaemia | Hyperinsulinaemia | Hyperglycaemia |
| glucose | Normoglycaemia | Hyperglycaemia | Insulin deficiency |

*SEM: standard error in the mean

Biochemical analysis reveals that the levels of free fatty acids in the plasma are greater in 10-week-old and 20-week-old ZLC rats, whereas the levels of free fatty acids in the liver of ZDF rats have decreased relative to the ZLC rats (FIG. 8A).

This period of 10 weeks is characterized by the separation of the weight gain curves for the ZDF rats and the ZLC rats, indicating the development of diabetes (FIG. 8B).

g) Effect of the Overexpression of FATP5 on the Acylation of Fatty Acids

The functional effects of the overexpression of hFATP5 in the primary hepatocytes of ZDF and ZLC rats were analysed.

The overexpression of FATP5 very significantly increases the acylation of fatty acids in the primary hepatocytes of ZDF rats, early at 5 minutes and late at 30 minutes.

These results are shown in FIG. 9.

h) Reversal of the Deficit of the Degradation of Fatty Acids in the Liver of Diabetic Fatty Rats

The beta-oxidation of fatty acids is deficient in the livers of ZDF rats compared with that which takes place in the livers of ZLC control rats. The overexpression of FATP5 reverses this deficit. The overexpression of FATP5 has no effect in the control rats. These results are shown in FIG. 10.

i) The Overexpression of FATP5 Reduces the Glucose and Lipid Level in the Blood of ZDF Rats

The glucose levels are significantly reduced at the age of 10 weeks in ZDF rats infected with the adenovirus carrying FATP5, compared with rats infected with the LacZ control adenovirus (FIG. 11C). This drop correlates with that of the free fatty acid levels (FIG. 11A) and with the large and rapid decline in the plasma triglyceride level (FIG. 11B).

This is proof that the expression of FATP5 affects the glucose metabolism and lowers the hyperglycaemia in vivo.

EXAMPLE 2

### Characterization of the Putative Promoter Region of the Human FATP5 Gene and Demonstration of the Promoter Activity

The FATP5 gene products (AF064255 or NM_{—}012254) belong to a family of proteins involved in lipid synthesis.

The FATP5 gene product is involved in the synthesis of complex lipids and especially in the elongation of fatty acids. The FATP5 gene is situated on chromosome 19.

The general information relating to the sequence coding for FATP5 is as follows (Table IV):

| Synonyms | SLC27A5, FATP5, VLACSR, |
|---|---|
| | VLCSH2, VLCS-H2 |
| Function | Ligase |
| Location | 19q13.43 |
| GenBank accession no. | NM 012254 |
| Size (bp) | 2347 |
| GenPept accession no. | NP 036386 |
| Size (amino acids) | 690 |

a) Location of the Transcription Start Sites (TSS) of FATP5 by Prediction, and Identification by the Technology of Rapid Amplification of Complementary DNA ends (Rapid Amplification of cDNA ends, RACE)

The FATP5 gene is situated on chromosome 19 and is composed of 10 exons distributed over 14 kb. The contig NT_{—}011104 containing the FATP5 gene was published in August 2002.

The 5′ UTR region mapped from the rnRNA and ESTs shows a size of 34 bp (5′UTR1). Thus the most probable “biological” transcription start site, determined relative to the 5′ end of the mRNA, of ESTs published by GenBank and information originating from DBTSS (Data Base of Human Transcriptional Start Sites), is situated as indicated in FIG. 12B.

b) Location of the FATP5 Promoter by Prediction

Materials and methods

Preparation of the RNAs of HepG2 cells:

The RNA is purified from one million HepG2 cells according to the Qiagen RNeasy mini kit protocol (Qiagen, Courtaboeuf, France).

5′ RACE:

The TSS are identified using the GeneRacer kit, catalogue no. L1500-01, L1500-02, L1502-01, L1502-02 version J05300225-0355, according to the supplier's recommendations.

Results:

A 10,000 bp region upstream from the start site, determined as indicated above, was selected from the contig NT_{—}011104. The analysed region stops just upstream from the coding region in position 10023, i.e. upstream from the ATG.

Predictions were made using softwares for the identification of putative promoter regions. These softwares—Promoter Inspector, First EF, etc.—each use a mathematical model constructed on the basis of the characteristics of the Polil eukaryotic promoters (TATA signals, GC composition bias, hierarchic organization, etc.). Softwares characterized by different approaches (quadratic discriminant analysis, Markov chain, etc.) and based on different characteristics make it possible to substantiate the predictions found in the same position.

The results of the most pertinent predictions are summarized in Table V below.

TABLE V
Predictions of promoters for the FATP5 gene

| | Position of | Position of | | |
|---|---|---|---|---|
| | predicted | predicted | | |
| Software | TSS | promoters | Other results | |
| Dragon | 7654, 7787, | | | |
| Promoter | 8055, 8443, | | | |
| | 8944 | | | |
| NNPP | | 8370-8420, | | |
| | | 8618-8668 | | |
| Promoter 2.0 | 9100 | 7458-7708 | | |
| Promoter | | 7458-7708 | | |
| Scan | | | | |
| TSSG/TSSW | 8939 | | TATA box: | |
| | | | 8911 | |
| Human Core | 8947, 8948 | | | |
| Promoter | 8949, 8950 | | | |
| Finder | | | | |
| CpG | | | | CpG islet: |
| report/plot | | | | 7620-8056 |
| Promoter | | 2021-2580 | | |
| Inspector | | | | |
| First EF | | 7409-7978, | 1st exon: | CpG islet: |
| | | 7577-8146 | 7909-8699 | 7685-7886 |
| | | | 1st exon: | CpG islet: |
| | | | 8077-8149 | 7685-7886 |

These predictions make it possible to locate the promoter of the human FATP5 gene within the region between 7400 and 10,000 bp, as determined by the extreme positions identified by the different softwares (FIG. 12A).

The promoter region ought to be between bases 8500 and 10,000 because another gene ends in position 8500 and 10,000 is the position of the ATP codon. In fact, the region upstream from the 8500 bp position corresponds to the NM_{—}032792 gene, whose function is unknown, and the region downstream from the 10,000 bp position corresponds to the FATP5 gene. This information should be compared with the promoter predictions made with the bioinformatic softwares, which located the promoter within the region between 7400 and 9100 bp.

This region situated upstream from exon 1 of the human FATP5 gene is assumed to contain the putative promoter or regulatory elements important for the regulation of this gene. The region analysed with the templates of Transfac (The Transcription Factor Database), and declared moderately conserved, extends between the limits 7400 and 10,023. The biological data of the EST and full-length mRNA type enabled the transcription start site (TSS) to be situated in position 9990.

Thus the sequence described in FIG. 13 corresponds to the putative promoter of the human FATP5 gene. This sequence is identified under the number SEQ ID no. 1 in the annexed sequence listing.

A TSS other than that in position 9990 could be identified in position 9928. Yet other TSS could be used as alternatives according to the cell types.

c) Analvsis of the Promoter Sequence of the Human FATP5 Gene Relative to the Prior Art of Patent WO 0121795

A series of searches were made for similarity between the sequence of the putative promoter of the human FATP5 gene, forming the subject of the invention, and the sequences contained in different accessible sequence banks such as “Htgs” (non-terminated sequences), “Chromosome” (complete genome, chromosomes and terminated contigs) and finally “nr” (all the published sequences).

A search for similarity between the genomic environment of the human FATP5 gene (contig NT_{—}011104) and the promoter region published in international patent application WO 01/21785 shows no sequence similarity.

A similarity search against the “Chromosome” subdivision of GenBank does not favour the emergence of a significant similarity with man or other species referenced in this data base (yeast, drosophila, etc.).

A similarity search among the sequences contained in the Htgs data base does not make it possible to characterize a human contig to which the promoter sequence of international patent application WO 01/21785 might correspond.

To confirm that the promoter sequence of international patent application WO 01/21785 shows no similarity with the genomic environment of the human FATP5 gene, these two sequences were aligned. No significant similarity is revealed.

Analysis of the promoter sequence of the FATP5 gene published in international patent application WO 01/21785 shows that this sequence does not correspond to the promoter of the human FATP5 gene, forming the subject of the invention, but does correspond to the promoter of the mouse FATP5 gene.

In conclusion, the different searches made allow the conclusion that the sequence of the promoter of the human FATP5 gene, forming the subject of the invention, has no significant similarity with any known sequence.

d) Confirmation of the Promoter Activity of the Nucleotide Sequence Forming the Subject of the Invention

Materials and methods

Amplification of the putative promoter sequence of FATP5:

Template: human placental genomic DNA (Sigma, St. Louis, Mo.);

Oligo sense (Invitrogen, Cergy Pontoise, France):
SEQ ID no. 3:
CGCTCGAGCTGTGAGCACCTGGATCAGTGCGTGCC;
Oligo antisense (Invitrogen, Cergy Pontoise,
France): SEQ ID no. 4:
CCCAAGCTTGGTACCAGCTCCTCCCTAGG;

Taq platinum Pfx+buffer 10X+MgSO₄ (Invitrogen, Cergy Pontoise, France);

dNTP (Promega, Williamsburg, Iowa).

Purification of the insert corresponding to the hFATP5 promoter: size of the insert=1573 bases.

Amplification programme:

1 incubation cycle at 94° C. for 5 minutes,

then 25 incubation cycles at 94° C. for 30 seconds, at 55° C. for 30 seconds, at 68° C. for 2 minutes.

The product obtained is purified with the QIAquick PCR purification kit (Qiagen, Courtaboeuf, France) and kept at 4° C.

Restrictions for cloning the promoter of the human FATP5 gene into vector pGL3 containing the luciferase gene:

| | R1 | R2 |
|---|---|---|
| pGL3 (Promega, Williamsburg, IA) | 5 μg | |
| Purified PCR product | | 41 μl |
| Buffer 2 10× (NEB, Beverly, MA), cat#B7002S | 5 μl | 5 μl |
| Xhol (NEB, Beverly, MA), cat#R0146S | 2 μl | 2 μl |
| HindIII (NEB, Beverly, MA), cat#R0104S | 2 μl | 2 μl |
| H₂O | 36 μl | |

Incubation for 2 h at 37° C.

Ligation according to the standard protocol of the Quick ligation kit (NEB, Beverly, Mass.), cat#M2200S.

Transformation according to the standard protocol of thermal shock for 30″ at 42° C.

Bacteria: DH5 alpha (Invitrogen, Cergy Pontoise, France).

Plating on dishes of LB agar (Invitrogen, Cergy Pontoise, France) containing 50 μg/ml of ampicillin.

Incubation overnight at 37° C.

Screening:

Each clone is subcultured on LB agar containing 50 μg/ml of ampicillin and is resuspended in 20 μl of water.

The suspension is heated for 10′ at 95° C.

PCR conditions for Selection of the DGL3 Clones/hFATP5 Promoter

Template: bacterial lysate

Oligo sense (Invitrogen, Cergy Pontoise, France):
SEQ ID no. 5:
TTCATTACATCTGTGTGTTGGTTTTTTGTGTG;
Oligo antisense (Invitrogen, Cergy Pontoise,
France): SEQ ID no. 6:
TATGCAGTTGCTCTCCAGCGGTTCCATCTTCC;

Goldstar+buffer 10X+MgCl₂ (Eurogentec, Seraing, Belgium), cat#ME-0064-50 μg/ml.

Amplification programme:

1 incubation cycle at 94° C. for 5 minutes,

then 25 incubation cycles at 94° C. for 30 seconds, at 55° C. for 30 seconds, at 72° C. for 2 minutes,

then 1 cycle at 72° C. for 7 minutes.

The product obtained is kept at 4° C.

Production of the plasmid according to the standard protocol of the EndoFree Plasmid maxi kit (Qiagen, Courtaboeuf, France, cat#12362).

Sequencing according to the standard protocol of the BigDye terminator V2.0 cycle sequencing kit (Applied Biosystems, Foster City, Calif., cat#4314415).

Results:

The 1573 base fragment of the human FATP5 promoter was cloned into pGL3 and sequenced. Constructions by successive deletions of the fragment will make it possible to identify the promoter region specific for FATP5 hepatic expression.

Transient transfection of HEP2, HELA and HEK293 hepatocytes:

Transiently transfected cells are used to identify the smallest possible promoter fragment for screening. Transfection is carried out by the jetPEI protocol (Polytransfection, llikirch, France) according to the supplier's recommendations.

On day 3: Luminometer reading:

The medium containing the transfection mixture is removed.

The plates are rinsed twice with 250 μl of PBS, and 100 μl of lysis buffer diluted to 1/5 are added. The plates are incubated for 30 min at room temperature, with shaking. 10 μl of lysate from each well are distributed into a Greiner 96-well white plate (Greiner Bio-One, Frickenhausen, Germany).

The luciferase activity is measured using the Dual Luciferase kit (Promega, Madison, Wiss., USA). The reading is taken with a TR717 Microplate Luminometer (Applied Biosystems, Foster City, Calif., USA).

The luciferase luminescence results are normalized against renilla and expressed as the induction of the construction containing the promoter, relative to void vector pGL3.

Results:

The promoter activity of the entire fragment of the FATP5 promoter is confirmed. These results, shown in FIG. 14, confirm that the nucleic acid sequence is indeed the FATP5 promoter and that it is functional. The highest values of luciferase activity are observed in HepG2 human hepatic cells.

### EXAMPLE 3

### Screening Test on Cells Stably Expressing the Human FATP5 Promoter

a) Cloning of the Putative Promoter of the Human FATP5 Gene into Vector PGL3 Hygromycin with Hygromycin Resistance

Production of vector pGL3 hygromycin:

The gene coding for hygromycin under the control of the TK promoter (size: 1671 bases) was amplified from vector pREP4 (Invitrogen, Cergy Pontoise, France) with the aid of the following primers:

Materials and method:

Oligo sense (Invitrogen, Cergy Pontoise, France):
SEQ ID no. 7:
GAAGATCTCTGCTTCATCCCCGTGGC;
Oligo antisense (Invitrogen, Cergy Pontoise,
France): SEQ ID no. 8:
GAAGATCTACCAGACCCCACGCAACGC;

Goldstar+buffer 10 X+MgCl₂ (Eurogentec, Seraing, Belgium).

The amplification programme and the purification of the product obtained are carried out according to protocols identical to those previously used for the amplification and purification of the insert corresponding to the hFATP5 promoter.

The fragment obtained is cloned into vector pGL3-Basic (Promega, Williamsburg, Iowa) at the Bglll site. The restriction for cloning, the ligation and the transformation are carried out according to protocols identical to those previously used for the amplification and purification of the insert corresponding to the hFATP5 promoter.

The clones which have integrated plasmid pGL3 with the TK hygromycin fragment are selected by PCR according to a protocol identical to that used for selection of the pGL3 clones/hFATP5 promoter using, as template, a bacterial lysate and:

Oligo sense (Invitrogen, Cergy Pontoise, France):
SEQ ID no. 9:
CTGCTTCATCCCCGTGGC;
Oligo antisense (Invitrogen, Cergy Pontoise,
France): SEQ ID no. 10:
ACCAGACCCCACGCAACGC;

Goldstar+buffer 10 X+MgCl₂ (Eurogentec, Seraing, Belgium).

The selection of the pGL3 hygromycin clones containing the hFATP5 promoter, the production of the plasmids and the sequencing are carried out according to protocols identical to those used for selection of the pGL3 clones/hFATP5 promoter.

b) Stable Transfection of HePG2 Heratocytes

The media used are described in the Table below.

| | Initial | | Final | Sup- |
|---|---|---|---|---|
| Culture medium | concentration | | concentration | plier |
| Dulbecco modified | | | | Gibco |
| Eagle's high glucose | | | | |
| medium | | | | |
| FCS (foetal calf | | | 10% | Gibco |
| serum) inactivated at | | | | |
| 56° C. for 30′ | | | | |
| Penicillin/ | 10,000 | U/ml | 0.125% | Biochrom |
| streptomycin | | | | AG |
| Sodium pyruvate MEM | 100 | mM | 1% | Gibco |
| Dulbecco's PBS | | | | Gibco |
| without Ca, Mg, Na | | | | |
| bicarbonate | | | | |
| Trypsin-EDTA | | | | Gibco |
| L-glutamine | 200 | mM | 1% | Gibco |

(100×)

c) Plating of the Cells

A culture of HepG2 cells is prepared. The cells are dissociated when they reach 80% confluence and are reinoculated into the culture medium in a 6-well plate at a rate of 320,000 cells per well. The cells are incubated for 24 h at 37° C., 5% CO₂.

d) Transfection and Activation

Transfection is carried out by the jetPEI protocol (Polytransfection, IIIkirch, France) with 500 ng of the plasmid prepared in a), according to the supplier's recommendations.

The medium of cells cultivated in a 6-well plate is aspirated. 1 ml of serum-free DMEM and 80 ,μl of transfection mixture are deposited in each well. The cells are then left to stand for 2 hours at 37° C. under an atmosphere containing 5% of C0₂. When these two hours have elapsed, 1 ml of FCS-free culture medium and Ultroser SF (USF, BioSepra, Cergy Pontoise, France) at a final concentration of 2% are added.

e) Selection of the Transfected Cells

The medium in each well is aspirated after 48 hours of culture. 2 ml of culture medium containing 10% of FCS and hygromycin at a final concentration of 0.2 mg/ml are added. The cells are dissociated when they reach 80% confluence.

The cells from 2 wells are then mixed and inoculated into the same selection medium in a 150 mm Petri dish. The selective culture is maintained until cellular clones appear.

f) Isolation of the Clones

When the clones are clearly individualized, they are isolated from the rest of the culture with the aid of cylinders. The cells they contain are then rinsed with PBS and removed after aspiration of the medium.

The cells are placed in a 96-well plate and 250 μl of DMEM containing 10% of FCS and hygromycin at a final concentration of 0.25 mg/ml are added. Culture is continued to 80% confluence.

g) Screening of the HepG2 Cells Stably Expressing the Human FATP5 Promoter

The clones are amplified in order to prepare a stock.

The cells derived from each clone are cultivated to 80% confluence in DMEM containing 10% of FCS, a penicillin/streptomycin mixture (0.125%) and 0.25 mg/ml of hygromycin, in a 24-well plate.

The treatments with the test compounds are applied for a period of 24 hours. When these 24 hours have elapsed, the luciferase activity is measured on the cell lysate using an Applied Biosystems TR717 luminescence reader (California, Calif.).

The cells exhibiting luciferase activity show that this is induced by the putative promoter contained in the plasmid they have received.

This result shows that the nucleic acid of the invention is indeed a promoter capable of directing the expression of a gene placed under its dependence.

### EXAMPLE 4

### Reversal of the Diabetic Phenotype and Regulation of the Expression of the FATP5 Gene in the ZDF Rat by an Insulin Sensitizer

| Reagent | Catalogue no. | Supplier |
|---|---|---|
| Dulbecco's medium without | 11880-028 | Gibco BRL |
| phenol red | | |
| Dialysed foetal bovine serum | 10110-161 | Gibco BRL |
| Glutamine | B-3000D | HyQ |
| Penicillin/streptomycin | 3-3001-D | HyQ |
| mixture (10,000 U/ml-10,000 | | |
| μg/ml) | | |
| Cell dissociation solution | C 5914 | Sigma |
| L-proline | P-4655 | Sigma |
| Phenol red | P-5530 | Sigma |
| C1-Bodipy-C12 (probe) | | Molecular Probes |

Agonists of the RXR nuclear receptor, called rexinoids, are insulin sensitizers and have beneficial effects on diabetic fatty animal models (type 2 diabetes). The bexarotene Targretine® (CAS 1543559-49-0) selectively activates nuclear receptor heterodimers: RXR-PPAR (peroxisome proliferator-activated receptor) or RXR-LXR (liver X receptor).

Male diabetic fatty rats (Zucker diabetic fatty rats (ZDF rats)) are kept in an animal house with free access to food and water, the nyctohemeral rhythm being observed.

The compound LGD 1069 is added to the food at a dose of 0.3, 1.0, 3.0, 10 or 30 mg/kg/day and administered for 50 days as from the age of 7 weeks. 4 animals are fed without the addition of LGD 1069: 2 ZDF and 2 ZLC. The animals are fasted and sacrificed the next day. The tissues are collected and frozen immediately in liquid nitrogen.

The hepatic RNAs are extracted by the conventional technique derived from Chomczynski and Sacchi. The expression is analysed by Northern blotting with hybridization using a radioactive cDNA probe specific for FATP5.

The results are shown in FIG. 15.

Negative regulation of the expression of FATP5 in the liver of ZDF rats compared with ZLC rats is counteracted by a treatment with the RXR agonist, which reverses the development of diabetes.

### EXAMPLE 5

### Test for Measuring the Active Transport of Fatty Acids Via the FATP5 Receptor Into Eukaryotic Cells (CHO: Chinese Hamster Ovary cells)

The use of a fluorescently labelled fatty acid mimicking molecule affords rapid measurement, in a microtitre plate format, of the intracellular accumulation of fatty acids in cells that overexpress the FATP receptor.

Description of the Test t,0360

Specialized Equipment Required
CO₂ incubator;
Microscope;
Multidrop;
Microplate washer;
Fluorimeter for microplates with fluorescein-type filters.

Protocol

Day 1

Distribution of the Cells Into 96-well Microplates

CHO eukaryotic cells that overexpress the FATP receptor are distributed into 96-well culture plates at a density of about 10,000 cells per well according to the following procedure:
Under a laminar flow hood, 4 ml of cell dissociation solution are added at 37° C. to a 225 cm³ culture flask.
The solution is shaken by hand over the cellular monolayer with an orbital rotary movement for 10-15 seconds, this being followed by aspiration with a 10 ml serological pipette.
4 ml of cell dissociation solution are added and the flask is put back in the incubator for 10 min (at most). After 10 minutes the detachment of the cells is checked under the microscope. The operation is continued until the cells are totally detached.
8 ml of medium are added to the flask and the contents are mixed from top to bottom several times in order to break up the clumps of cells. This cellular mixture is added to 88 ml of medium preheated to 37° C.

Preparation of the Multidrop
The Multidrop is placed in the laminar flow hood. The Multidrop head and the tube are cleaned by pumping 20-30 ml of 70% ethanol, then 20-30 ml of sterile water and finally 20-30 ml of medium.
The Multidrop tube is placed in the flask containing the cells and approximately 5-10 ml of medium are removed. 100 μl of cells are added per well (10,000 cells).
The microplates are placed in the incubator at 37° C., 10% CO₂, for 48 hours.

Day 2: Addition of the Test Molecules

The test molecules will be added according to the following plate layout:

| | | | | | | | | | | | |
|---|---|---|---|---|---|---|---|---|---|---|---|
| PC | T | T | T | T | T | T | T | T | T | T | NC |
| PC | T | T | T | T | T | T | T | T | T | T | NC |
| PC | T | T | T | T | T | T | T | T | T | T | NC |
| PC | T | T | T | T | T | T | T | T | T | T | NC |
| PC | T | T | T | T | T | T | T | T | T | T | NC |
| PC | T | T | T | T | T | T | T | T | T | T | NC |
| PC | T | T | T | T | T | T | T | T | T | T | NC |
| PC | T | T | T | T | T | T | T | T | T | T | NC |

Using the Multidrop, 95 μl of DMEM (without phenol red) are distributed into each well of a 96-well microplate.
The test molecules are simultaneously distributed at a final concentration of 5 mg/ml (1% of DMSO) into the wells marked T.
200 mM lauric acid is distributed into the negative control wells (NC).
5 μl of PBS are distributed into the positive control wells (PC).

Day 3
The cells are washed twice with PBS.
The cells are incubated for 2 minutes at 37° C. in a fatty acid mimicking solution containing 0.1 mM BODIPY-FA and 0.1% of BSA without fatty acids, in PBS.
After 2 minutes the cells are washed four times with a PBS/0.1% BSA mixture placed in ice beforehand.
The plate is read on a fluorescence microplate reader using 485 nm as the excitation wavelength and 530 nm as the emission wavelength.

The results will be expressed as a percentage of the control cells without probe (PC) in arbitrary fluorescence units.

This test makes it possible to measure the active transport of fatty acids via the FATP5 receptor into eukaryotic cells. It also makes it possible to measure the effect of a given molecule on the active transport of fatty acids via the FATP5 receptor and thereby to determine its ability to stimulate or inhibit the activity of the promoter of the human FATP5 gene.

1) Isolated human nucleic acid sequence, characterized in that it comprises at least the nucleotide sequence identified under the number SEQ ID no. 1 in the annexed sequence listing.

2) Isolated human nucleic acid sequence, claim 1 characterized in that it consists of the nucleotide sequence identified under the number SEQ ID no. 1 in the annexed sequence listing.

3) Nucleic acid sequence according to claim 1, characterized in that it codes for the human FATP5 protein.

4) Nucleic acid sequence according to claim 3, characterized in that it corresponds to the nucleotide sequence identified under the number SEQ ID no. 2 in the annexed sequence listing.

5) Vector, characterized in that it comprises a nucleic acid sequence as described in claim 1.

6) Vector, characterized in that it comprises a nucleic acid sequence as described in claim 1.

7) Vector, characterized in that it comprises a nucleotide sequence of claim and one coding for a detectable protein placed under the dependence of the nucleic acid sequence of claim 1.

8) Vector according to claim 7, characterized in that the detectable protein is a protein selected from FATP5, green fluorescent protein (GFP), renilla and luciferase.

9) Vector according to claim 5 characterized in that it is a plasmid.

10) Host cell into which the nucleic acid sequence according to claim 1 has been introduced.

11) Cell according to claim 10, characterized in that it is a prokaryotic or eukaryotic cell.

12) Prokaryotic cell according to claim 11, characterized in that it is a bacterium.

13) Method of producing a protein, characterized in that a host cell as described in claim 10 is cultivated under conditions that allow the expression, in the form of a protein, of the nucleotide sequence which has been introduced into said cell.

14) Use of the nucleic acid sequence as described in claim 1 for the identification of a product capable of modulating the expression of a nucleotide sequence placed under the dependence of the nucleic acid sequence as described in claim 1.

15) Method of identifying a product capable of modulating the expression of a nucleotide sequence placed under the dependence of the nucleic acid sequence as described in claim 1, characterized in that
a) a nucleic acid sequence consisting of that of claim 1 is brought into contact, in a medium suitable for the expression of a nucleotide sequence, with a product capable of modulating the expression of a nucleotide sequence placed under the dependence of the nucleic acid sequence as described in claim 1; and
b) the level of expression of said nucleotide sequence placed under the dependence of the nucleic acid sequence as described in claim 1 is measured by any appropriate means.

16) Method according to claim 15, characterized in that it comprises an additional step for comparison of the level of expression determined in b) with the level of expression of said nucleotide sequence placed under the dependence of said nucleic acid sequence, in a control which has not been brought into contact with the product to be identified.

17) Method of identifying a product capable of interacting with a nucleic acid sequence as described in claim 1, characterized in that
a) a nucleic acid sequence as described in claim 1 is brought into contact with a product capable of interacting with the nucleic acid sequence as described in claim 1; and
b) the interaction between the nucleic acid sequence as described in claim 1 and the test product is evaluated.

18) Method of identification according to claim 17, the purpose of which is to identify a product capable of binding to said nucleic acid sequence.


SEQUENCE LISTING (22 sequences)

SEQ ID NO: 1 - DNA, 1574 bp, Homo sapiens
gctgtgagcacctggatcagtgcgtgccttgccctcatagacaagatgtgagtgtcagggttcagcgtccctgttcttgggtggcatccatgcggcccacaggctgtcctgggcaggaacaggatggtccactttgccatctaggacctctattctgtaggggctaaggtgggctgcatatataaccagcacttggttctcttggatccctcagtcctgtggggagtaggataggcaggccacatctgaggtgggtctccgagagggaggccacgtgaaagccccatcatactcctggttgctgctgtccacgacctttaaggcccctcccatagatcttccagtcaggctctaagggtcttttggacttaagacttcccctccccgaccttgatggacatggctgagcaccttacttgtggggagcccagggggatctttggggccaggttgcactgagggtatataagcccccatatatctgcccttggtgctgggggcaaagttcagggagggctccgatggccgtgttgggggcaggaaacagtgaggggtgggatcctggggctgactgctgggcggttggggacaccacccctactggccagccagtgtcccaggcccctgcatccagcggagagagcagccactctttgcccctctcctcttcagcacaatagtggctcctagcctaggtgccctgccataaggatttggagctggtggagtggggtatggccccaggccataccccagcctgacctgccaggctcctaggccaatggcagctcctctgggttcaccttctgagactgcttcctcaaggtgagaggccagggctttcatgatccaactgtgtgcatcatgggctgacttcagtctcctgaaccccaggactccatcgtcgtcaaagccagaggtggagcagagagtcagagcaagcacatgggtggggggcagagctgggtttgaatccaggcgatgccagtgttcctgtatttactgagcacttgctgtatgcctggcaggagggtacgattcctcggatcatgggacaaggggcttagcctggctgagcccctctcctcagggtttcagtgggtgtcacaggaggacctcccaatgaggtccaccttgctgtcctgttgcttttctgtttggcctgattggagtgttcccaagggcagggatttctgtgctctgagtgtctggtacccagcagggatgggaggggaggcaagggagttagatgccagccctggagggtggatggggcaagcaggacagcaggaaggaggctagtggtgcccaaggggcagagacctgggaacccagaggccagtgatggaaagggttagcgccaggcagagaggggctggacccacacacaagaccaggcagagcaaacaaacgtagaggagaagggctgggggcaaagtcctggggagaggaccagccactgtcagcagtgccagggccaccacccctggggaggagccattgggctactcttgaggggccagaggtgagcctgaacactccacagctgctgctcctagggaggagctggtacc

SEQ ID NO: 2 - DNA, 3647 bp, Homo sapiens
gctgtgagcacctggatcagtgcgtgccttgccctcatagacaagatgtgagtgtcagggttcagcgtccctgttcttgggtggcatccatgcggcccacaggctgtcctgggcaggaacaggatggtccactttgccatctaggacctctattctgtaggggctaaggtgggctgcatatataaccagcacttggttctcttggatccctcagtcctgtggggagtaggataggcaggccacatctgaggtgggtctccgagagggaggccacgtgaaagccccatcatactcctggttgctgctgtccacgacctttaaggcccctcccatagatcttccagtcaggctctaagggtcttttggacttaagacttcccctccccgaccttgatggacatggctgagcaccttacttgtggggagcccagggggatctttggggccaggttgcactgagggtatataagcccccatatatctgcccttggtgctgggggcaaagttcagggagggctccgatggccgtgttgggggcaggaaacagtgaggggtgggatcctggggctgactgctgggcggttggggacaccacccctactggccagccagtgtcccaggcccctgcatccagcggagagagcagccactctttgcccctctcctcttcagcacaatagtggctcctagcctaggtgccctgccataaggatttggagctggtggagtggggtatggccccaggccataccccagcctgacctgccaggctcctaggccaatggcagctcctctgggttcaccttctgagactgcttcctcaaggtgagaggccagggctttcatgatccaactgtgtgcatcatgggctgacttcagtctcctgaaccccaggactccatcgtcgtcaaagccagaggtggagcagagagtcagagcaagcacatgggtggggggcagagctgggtttgaatccaggcgatgccagtgttcctgtatttactgagcacttgctgtatgcctggcaggagggtacgattcctcggatcatgggacaaggggcttagcctggctgagcccctctcctcagggtttcagtgggtgtcacaggaggacctcccaatgaggtccaccttgctgtcctgttgcttttctgtttggcctgattggagtgttcccaagggcagggatttctgtgctctgagtgtctggtacccagcagggatgggaggggaggcaagggagttagatgccagccctggagggtggatggggcaagcaggacagcaggaaggaggctagtggtgcccaaggggcagagacctgggaacccagaggccagtgatggaaagggttagcgccaggcagagaggggctggacccacacacaagaccaggcagagcaaacaaacgtagaggagaagggctgggggcaaagtcctggggagaggaccagccactgtcagcagtgccagggccaccacccctggggaggagccattgggctactcttgaggggccagaggtgagcctgaacactccacagctgctgctcctagggaggagctggtaccatgggtgtcaggcaacagttggccttgctgctgctgctgctgctcctgctctggggcctggggcagccagtgtggccagtcgctgtggccttgaccctgcgctggctcctgggggatcccacatgttgcgtgctacttgggctggccatgttagcacggccctggctcggcccctgggtgccccatgggctgagcctggcagctgcggccctggcactaaccctcctgccagcacggctgcccccaggactacgctggctgccggctgatgtgatcttcttggccaagatcctccacctgggcctgaagatcaggggatgcttgagccggcagccgcctgacacctttgtagatgccttcgagcggcgagcacgagcgcagcctggcagggcactcttggtgtggacggggcctggggccggctcagtcacctttggtgagctggatgcccgggcctgccaggcggcatgggccctgaaggctgagctgggtgaccctgcgagcctgtgtgccggggagcctactgccctccttgtgctggcttcccaggccgttccagccctgtgtatgtggctggggctggccaagctgggctgcccaacagcctggatcaacccgcatggccgggggatgcccctggcgcactctgtgctgagctctggggcccgggtgctggtggtggacccagacctccgggagagcctggaggagatccttcccaagctgcaggctgagaacatccgctgcttctacctcagccatacctcccctacaccaggggtgggggctctgggggctgccctggatgcagcgccctcccacccagtgcctgctgacctgcgtgctgggatcacatggagaagccctgccctcttcatctatacctcggggaccactggcctcccgaagccagccatcctcacgcatgagcgggtactgcagatgagcaagatgctgtccttatctggggccacagctgatgatgtggtttacacggtcctgcctctgtaccacgtgatgggacttgtcgttgggatcctcggctgcttagatctcggagccacctgtgttctggcccccaagttctctacttcctgcttctgggatgactgtcggcagcatggcgtgacagtgatcctgtatgtgggcgagctcctgcggtacttgtgtaacattccccagcaaccagaggaccggacacatacagtccgcctggcaatgggcaatggactacgggctgatgtgtgggagaccttccagcagcgcttcggtcctattcggatctgggaagtctacggctccacagaaggcaacatgggcttagtcaactatgtggggcgctgcggggccctgggcaagatgagctgcctcctccgaatgctgtccccctttgagctggtgcagttcgacatggaggcggcggagcctgtgagggacaatcagggcttctgcatccctgtagggctaggggagccggggctgctgctgaccaaggtggtaagccagcaacccttcgtgggctaccgcggcccccgagagctgtcggaacggaagctggtgcgcaacgtgcggcaatcgggcgacgtttactacaacaccggggacgtactggccatggaccgcgaaggcttcctctacttccgcgaccgcctcggggacaccttccgatggaagggcgagaacgtgtccacgcacgaggtggagggcgtgttgtcgcaggtggacttcttgcaacaggttaacgtgtatggcgtgtgcgtgccaggttgtgagggtaaggtgggcatggctgctgtgcagctagcccccggccagactttcgacggggagaagttgtaccagcacgttcgcgcttggctccctgcctacgctaccccccatttcatccgcatccaggacgccatggaggtcaccagcacgttcaaactgatgaagacccggttggtgcgtgagggcttcaatgtggggatcgtggttgaccctctgtttgtactggacaaccgggcccagtccttccggcccctgacggcagaaatgtaccaggctgtgtgtgagggaacctggaggctctga

SEQ ID NO: 3 - DNA, 35 bp, Artificial Sequence
  Description of Artificial Sequence Synthetic primer
cgctcgagctgtgagcacctggatcagtgcgtgcc

SEQ ID NO: 4 - DNA, 29 bp, Artificial Sequence
  Description of Artificial Sequence Synthetic primer
cccaagcttggtaccagctcctccctagg

SEQ ID NO: 5 - DNA, 32 bp, Artificial Sequence
  Description of Artificial Sequence Synthetic primer
ttcattacatctgtgtgttggttttttgtgtg

SEQ ID NO: 6 - DNA, 32 bp, Artificial Sequence
  Description of Artificial Sequence Synthetic primer
tatgcagttgctctccagcggttccatcttcc

SEQ ID NO: 7 - DNA, 26 bp, Artificial Sequence
  Description of Artificial Sequence Synthetic primer
gaagatctctgcttcatccccgtggc

SEQ ID NO: 8 - DNA, 27 bp, Artificial Sequence
  Description of Artificial Sequence Synthetic primer
gaagatctaccagaccccacgcaacgc

SEQ ID NO: 9 - DNA, 18 bp, Artificial Sequence
  Description of Artificial Sequence Synthetic primer
ctgcttcatccccgtggc

SEQ ID NO: 10 - DNA, 19 bp, Artificial Sequence
  Description of Artificial Sequence Synthetic primer
accagaccccacgcaacgc

SEQ ID NO: 11 - DNA, 29 bp, Artificial Sequence
  Description of Artificial Sequence Synthetic primer
gactacctcatgaagatcctgactgagcg

SEQ ID NO: 12 - DNA, 26 bp, Artificial Sequence
  Description of Artificial Sequence Synthetic primer
ggggcaatgatcttgatcttcatggt

SEQ ID NO: 13 - DNA, 20 bp, Artificial Sequence
  Description of Artificial Sequence Synthetic primer
ccacataccaggaaatgagc

SEQ ID NO: 14 - DNA, 20 bp, Artificial Sequence
  Description of Artificial Sequence Synthetic primer
gacatcaagaaggtggtgaa

SEQ ID NO: 15 - DNA, 35 bp, Artificial Sequence
  Description of Artificial Sequence Synthetic primer
gctctagagctggtaccatgggtgtcaggcaacag

SEQ ID NO: 16 - DNA, 35 bp, Artificial Sequence
  Description of Artificial Sequence Synthetic primer
cggaattcgctggtaccatgggtgtcaggcaacag

SEQ ID NO: 17 - DNA, 38 bp, Artificial Sequence
  Description of Artificial Sequence Synthetic primer
ggggtaccaatgccatgggtatttggaagaaactaacc

SEQ ID NO: 18 - DNA, 28 bp, Artificial Sequence
  Description of Artificial Sequence Synthetic primer
gggaactggacttcgggcaaatgtgtgg

SEQ ID NO: 19 - DNA, 39 bp, Artificial Sequence
  Description of Artificial Sequence Synthetic primer
gctctagatcagagcctccaggttccctcacacacagcc

SEQ ID NO: 20 - DNA, 33 bp, Artificial Sequence
  Description of Artificial Sequence Synthetic primer
gctctagatggtaccatgggtgtcaggcaacag

SEQ ID NO: 21 - DNA, 127 bp, Homo sapiens
gaccagccactgtcagcagtgccagggccaccacccctggggaggagccattgggctactcttgaggggccagaggtgagcctgaacactccacagctgctgctcctagggaggagctggtaccatg

SEQ ID NO: 22 - DNA, 127 bp, Homo sapiens; identical to SEQ ID NO: 21